(19) United States
(12) Patent Application Publication
(10) Pub. No.: US 2020/0297238 A1
(43) Pub. Date: Sep. 24, 2020

(54) APPARATUSES AND METHODS FOR ASSISTING, CONFIRMING, AND MONITORING PLACEMENT OF CATHETERS IN PATIENTS

(71) Applicant: The Board of Trustees of the Leland Stanford Junior University, Stanford, CA (US)

(72) Inventor: Chi-ho B. Tsui, Palo Alto, CA (US)

(73) Assignee: The Board of Trustees of the Leland Stanford Junior University, Stanford, CA (US)

(21) Appl. No.: 16/821,868

(22) Filed: Mar. 17, 2020

Related U.S. Application Data
(60) Provisional application No. 62/821,964, filed on Mar. 21, 2019.

(51) Int. Cl. A61B 5/06; A61B 5/042; A61B 5/0408; A61B 5/00; A61B 5/044; A61B 5/04

(52) U.S. Cl. CPC A61B 5/061; A61B 5/042; A61B 5/0408; A61B 5/6886; A61B 5/7405; A61B 2562/17; A61B 5/6833; A61B 5/6852; A61B 5/04012; A61B 5/6844; A61B 2503/045; A61B 5/044

(57) ABSTRACT

A medical method performed by a medical device, includes: generating a ECG tracing using a first electrode placed on a surface of a patient and a second electrode at a catheter inside the patient; and outputting the ECG tracing for presentation to a user; wherein the first electrode is fixed in position with respect to the patient; and wherein an amplitude of the ECG tracing corresponds with a relative distance between the first electrode and the second electrode. A medical device includes: a first electrode configured for placement on a surface of a patient; a second electrode configured for coupling with a catheter; and a processing unit configured to generate a ECG tracing using the first electrode on the surface of the patient and the second electrode inside the patient.

### RELATED APPLICATION DATA

This application claims priority to and the benefit of U.S. Provisional Patent Application No. 62/821,964 filed on Mar. 21, 2019, pending. The entire disclosure of the above application is expressly incorporated by reference herein.

### FIELD

One or more embodiments described herein relate to medical devices and methods for monitoring catheters, and more specifically to medical devices and associated methods for assisting, confirming, and monitoring placement of catheters in patients using ECG tracings.

### BACKGROUND

Umbilical venous catheters (UVC) are routinely inserted during the treatment of ill neonates for the administration of fluids and medication, as well as for central venous pressure monitoring. The catheters are inserted and blindly advanced from the umbilicus into the thorax. The optimal location for the tip of the venous catheter is in the inferior vena cava (IVC) or at the junction of the IVC and the right atrium (RA). The reported incidence of misplaced umbilical venous or arterial catheters is in the range of 2037%. If the tip is placed too high in the RA, there is an increased risk of myocardial infiltration and arrhythmias. If the tip is placed too low in the IVC, there may be a risk of either intrahepatic or extrahepatic placement into the portal vessels resulting in complications such as portal vein thrombosis, portal hypertension, and liver necrosis. Although radiographs are routinely used to confirm the proper positioning of umbilical arterial or venous catheters, this technique requires the movement of infants as well as radiation exposure to infants who are often critically ill. Therefore, an easy, reliable, and real-time guidance and confirmation technique for UVC placement would be beneficial. More importantly, studies have demonstrated that 50% of UVCs in infants born less than 32 weeks gestation migrated within the first week after insertion. Thus, it is critical to monitor the location of umbilical catheters.

### SUMMARY

A medical method performed by a medical device, includes: generating a ECG tracing using a first electrode placed on a surface of a patient and a second electrode at a catheter inside the patient; and outputting the ECG tracing for presentation to a user; wherein the first electrode is fixed in position with respect to the patient; and wherein an amplitude of the ECG tracing corresponds with a relative distance between the first electrode and the second electrode.

Optionally, the catheter comprises an umbilical venous catheter.

Optionally, the patient comprises a pediatric patient.

Optionally, the amplitude of the ECG tracing is equal to zero or has a minimum value when the second electrode is directly beneath the first electrode.

Optionally, the ECG tracing is outputted for display on a screen.

Optionally, the first electrode is located above a target position for the second electrode.

Optionally, the method further includes generating an audio signal when the second electrode is underneath the first electrode.

Optionally, the method further includes repeating the act of generating and the act of outputting while the catheter is being positioned inside the patient.

Optionally, the repeating is performed to provide a real-time indication of a position of the catheter with respect to the first electrode.

Optionally, the method further includes repeating the act of generating and the act of outputting after the catheter has been placed at a target position inside the patient.

Optionally, the repeating is performed to allow monitoring of the placed catheter at the target position.

Optionally, the method further includes generating an audio signal when the catheter is displaced from the target position.

A medical device includes: a first electrode configured for placement on a surface of a patient; a second electrode configured for coupling with a catheter; and a processing unit configured to generate a ECG tracing using the first electrode on the surface of the patient and the second electrode inside the patient.

Optionally, the medical device further includes a screen for displaying the ECG tracing.

Optionally, the processing unit is a part of a ECG device.

Optionally, the catheter comprises an umbilical venous catheter, and the second electrode is configured for coupling with the umbilical venous catheter.

Optionally, the processing unit is configured to generate the ECG tracing with an amplitude that is equal to zero or has a minimum value when the second electrode is directly beneath the first electrode.

Optionally, the medical device further includes a screen, wherein the processing unit is configured to output the ECG tracing for display on the screen.

Optionally, the first electrode is configured for placement above a target position for the second electrode.

Optionally, the medical device further includes a speaker configured to provide an audio signal when the second electrode is underneath the first electrode.

Optionally, the processing unit is configured to generate additional ECG tracing(s) while the catheter is being positioned inside the patient.

Optionally, the additional ECG tracing(s) provides a real-time indication of a position of the catheter with respect to the first electrode.

Optionally, the processing unit is configured to generate additional ECG tracing(s) after the catheter has been placed at a target position inside the patient.

Optionally, the additional ECG tracing(s) provides monitoring of the placed catheter at the target position.

Optionally, the medical device further includes a speaker configured to provide an audio signal when the catheter is displaced from the target position.

Optionally, the first electrode is on a pad with an adhesive surface and a plurality of markers.

Optionally, first electrode is located on the pad in association with one of the markers.

Optionally, the markers are in a single row.

Optionally, the markers are arranged in rows and columns.

A medical device includes: a pad having an adhesive surface; a plurality of markers at the pad; and an external electrode in positional correspondence with a selected one of the markers, wherein the external electrode is configured to couple with a lead extending from an apparatus, the apparatus having a processing unit.

Optionally, the markers are in a single row.

Optionally, the markers are arranged in rows and columns.

Optionally, the medical device further includes a catheter electrode configured to couple with another lead extending from the apparatus.

Optionally, the medical device further includes the apparatus.

Optionally, the apparatus comprises a ECG device.

A medical device includes: a pad having an adhesive surface; a plurality of electrodes coupled to the pad; and a plurality of terminals respectively at, or electrically connected to, the plurality of electrodes; wherein each of the terminals in the plurality of terminals is capable of being detachably coupled to a lead extending from an apparatus, the apparatus having a processing unit.

Optionally, the electrodes in the plurality of electrodes are in a single row.

Optionally, the electrodes in the plurality of electrodes are arranged in rows and columns.

Optionally, the medical device further includes a catheter electrode configured to couple with another lead extending from the apparatus.

Optionally, the medical device further includes the apparatus.

Optionally, the apparatus comprises a ECG device.

Optionally, the electrodes are radiopaque.

Optionally, the electrodes are coupled to respective markers.

A medical method includes: placing a pad on a surface of a patient, the pad having a plurality of markers; taking an image of the patient, the image indicating an internal anatomical structure of the patient and the plurality of markers; coupling an electrode with an apparatus via a lead, wherein the electrode is in positional correspondence with one of the plurality of markers that is the closest in position with respect to the internal anatomical structure of the patient as they appear in the image.

Optionally, the markers are in a single row.

Optionally, the markers are arranged in rows and columns.

Optionally, the apparatus comprises a ECG device.

Optionally, the method further includes using the apparatus to monitor a position of a catheter while the catheter is being positioned within the patient.

Optionally, the catheter comprises an umbilical venous catheter.

Optionally, the patient comprises a pediatric patient.

Other aspects, embodiments, and benefits will be described in the detailed description below.

### BRIEF DESCRIPTION OF THE DRAWINGS

The above and other features and advantages will become readily apparent to those skilled in the art by the following detailed description of exemplary embodiments thereof with reference to the attached drawings, in which:

FIG. 1 shows different ECG tracings for different anatomical structures.

FIG. 2A shows an example of a medical device.

FIG. 2B shows definition of bio-potential channels utilized in the medical device of FIG. 2A.

FIG. 2C shows a neonate with an external electrode (patch electrode) in place, wherein the external electrode is at a pad with adhesive, and a plurality of markers.

FIG. 2D shows an example of a radiograph with the radio-opaque markers and various components of a medical device in their respective operative positions.

FIG. 3A illustrates placement of electrodes in standard ECG.

FIG. 3B illustrates placement of electrodes in accordance with some embodiments.

FIGS. 4A-4D illustrates surface mapping in a patient and operation of the medical device of FIG. 2A.

FIG. 5 illustrates placement of device at lower abdomen of a patient.

FIG. 6 is a block diagram illustrating an embodiment of a specialized processing system.

### DETAILED DESCRIPTION

Various embodiments are described hereinafter with reference to the figures. Like reference numerals refer to like elements throughout. Like elements will, thus, not be described in detail with respect to the description of each figure. It should also be noted that the figures are only intended to facilitate the description of the embodiments. They are not intended as an exhaustive description of the claimed invention or as a limitation on the scope of the claimed invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

This disclosure describes devices and methods that provide an easy, reliable, and real-time confirmation technique to verify UVC placement, or placement of other catheters or medical devices, in patients.

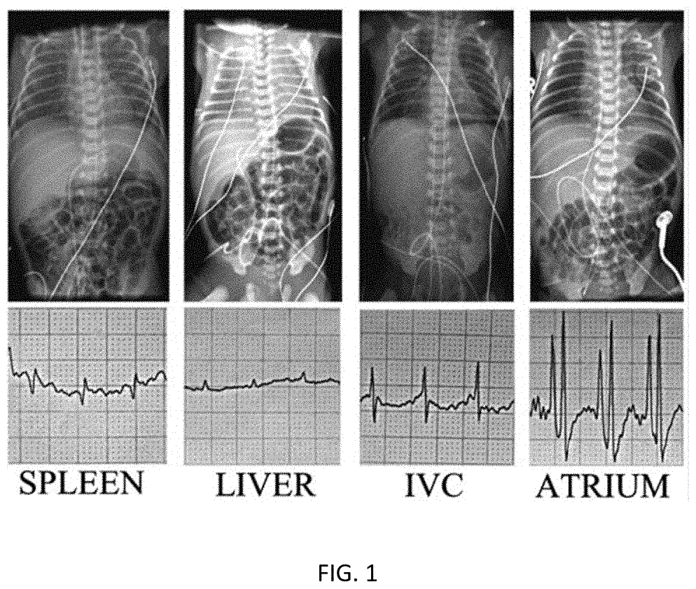

Electrocardiogram (ECG) may be used to guide the placement of UVC catheter (CVC) via a saline column. FIG. 1 shows different ECG tracings for different anatomical structures, such as spleen, liver, inferior vena cava, atrium, etc. Although the ECG method is able to avoid grossly misplacing catheters, small changes can be challenging to detect in some cases.

In some embodiments described herein, a medical device is provided that is able to improve on the guidance, detect small changes, and monitor catheter migration through reference to a target surface electrode, e.g., Target Surface UVC Indicator (TSUI) electrode.

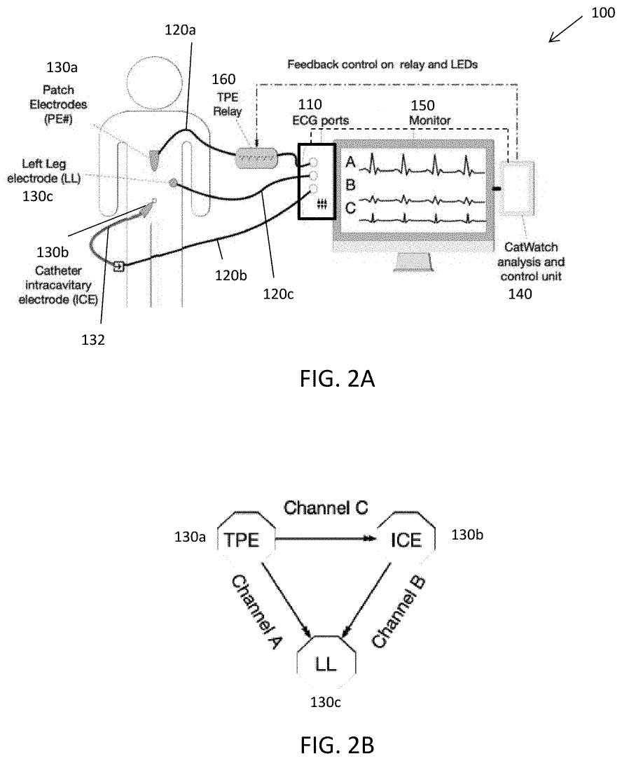

FIG. 2A shows an example of a medical device 100. The medical device 100 includes ports 110 with respective leads 120a-120c connecting to respective electrodes 130a-130c. In some embodiments, the ports 110 may be ECG ports. The first electrode 130a is a patch electrode (PE) configured for placement on a skin of a patient. The second electrode 130b is a catheter electrode (ICE) configured to be carried by a catheter 132, such as an UVC. The third electrode 130c (LL) is configured for placement at a patient's lower chest or leg. In other embodiments, the second electrode 130b may be configured to be carried by other types of medical devices that are not catheter. For example, in other embodiments, the second electrode 130b may be carried by an implant being delivered, a guidewire, etc. Also, in other embodiments, the third electrode 130c may be configured for placement at other locations that are not the lower chest or leg.

The first electrode 130a may be any electrode that is configured for placement on a patient. Similarly, the third electrode 130c may be any electrode that is configured for placement on the patient.

In some embodiments, the second electrode 130b may be an electrode secured to a part of the catheter 132. In other embodiments, the second electrode 130b may be an electrode on a separate device that is inserted into the catheter 132. In further embodiments, the catheter tip may be transformed into an electrode. In one specific implementation, prior to the insertion of the catheter 132 into a patient, a conductive Johans ECG adapter may be connected to a distal port of a 3 or 5 French (single or triple lumen) UVC. The catheter 132 may be primed with 0.9% normal saline to ensure an air-free column of fluid from the ECG connector to the catheter tip. This will transform the UVC catheter tip into a unipolar ECG electrode.

The medical device 100 also includes a processing unit 140. The processing unit 140 is configured to receive signals from the leads 120a-120c, and process the signals to generate graphic for display by a monitor 150. In the illustrated embodiments, the graphic are waveforms generated based on signals from the electrodes 130a-130c and/or based on channels defined among the electrodes 130a-130c. Examples of channels defined among the electrodes 130a-130c will be described with reference to FIG. 2B. In other embodiments, the graphic may have other configurations. For example, in other embodiments, the graphic may include a numerical value representing a distance between two electrodes (e.g., electrodes 130a, 130b) and/or a distance between the electrode 130b and a target location.

The processing unit 140 may be implemented using hardware, software, or a combination of both. In some embodiments, the hardware for implementing the processing unit 140 may include a processor, such as a general purpose processor, a microprocessor, a FPGA processor, an ASIC processor, or any of other types of processors. Also, in some embodiments, the hardware for implementing the processing unit 140 may include an integrated circuit. The processing unit 140 may include one or more processors and/or one or more modules in some embodiments. In some embodiments, the processing unit 140 may include an analysis and control unit. Also, in some embodiments, the processing unit 140 is configured to process signals from the electrodes 130a, 130b, 130c, and to interpret the signals. In addition, in some embodiments, the processing unit 140 may also be configured to generate output indicating a position of a catheter tip of the catheter 132.

Referring to FIG. 2A, the medical device 100 also includes a relay 160 coupled to the lead 120a. The relay 160 is configured to communicate with the processing unit 140 during use. The relay 160 will be described in further detail.

FIG. 2B shows definition of bio-potential channels (arrow points from + to 1) for the medical device 100. Channel A is defined as a channel between the first electrode 130a (e.g., PE) and the third electrode 130c (e.g., LL). Channel B is defined as a channel between the second electrode 130b (e.g., ICE) and the third electrode 130c (e.g., LL). Channel C is defined as a channel between the first electrode 130a (PE) and the second electrode 130b (e.g., ICE). In other embodiments, the direction for any of the above examples of channels may be opposite. Also, in other embodiments, the device 10 may not include all three channels A, B, C. For example, the device 10 may not include the channel A and/or the channel B in some embodiments.

FIGS. 2C-2D show an example of an electrode device 200 for implementing the first electrode 130a (patch electrode). The electrode device 200 is illustrated as being placed in position with respect to a neonate. In the illustrated embodiments, the electrode device 200 includes a pad 202 with adhesive for detachably coupling to a skin of the patient. The electrode device 200 also includes a plurality of markers 210, and a plurality of electrodes 250 (shown in FIG. 2D). One of the electrodes 250 may be selected to function as the first electrode 130a. As shown in FIG. 2C, the electrode device 200 is coupled to the relay 160. The relay controller 160 is an interface between the electrodes 250 and the processing unit 140. The relay controller 160 is configured to define, based on user input, which one of the electrodes 250 of the electrode device 200 is to be connected to the processing unit 140 of the medical device 100 at any given time without impeding other physiological measurements. In other embodiments, the relay controller 160 may be implemented on the pad 202 or at the processing unit 140.

As shown in FIG. 2C, the electrode device 200 also includes a series of LEDs 252 for visual feedback. In some embodiments, the LEDs 252 are configured to inform a user of the device 100 regarding a position of the catheter 132 inside the patient with respect to a target region in the patient. In one implementation, the LEDs 252 are arranged in a row, with a center one of the LEDs 252 representing a desired position of the end of the catheter 132 with respect to a target region in the patient. In such cases, when the end of the catheter 132 is at a desired position in the patient, the center LED 252 (e.g., first LED) will be on. When the end of the catheter 132 move slightly away from the desired position in one direction (e.g., a first direction), then the center LED 252 will be off, and another LED 252 (e.g., a second LED) adjacent to the center LED 252 will be on. If the end of the catheter 132 move further away from the desired position in the same direction, then the second LED will be off, and the adjacent LED (e.g., a third LED) will be on. On the other hand, when the end of the catheter 132 move slightly away from the desired position in another direction (e.g., a second direction opposite from the first direction), then the center LED 252 will be off, and another LED 252 (e.g., a fourth LED) adjacent to the center LED 252 will be on. If the end of the catheter 132 move further away from the desired position in the same direction (e.g., second direction), then the fourth LED will be off, and the adjacent LED (e.g., a fifth LED) will be on. In some embodiments, the processing unit 140 is configured to process signals from the electrodes 130, and to generate a control signal to cause one of the LEDs 252 to illuminate. For example, the processing unit 140 may be configured to analyze waveform in Channel C, and to determine a distance between the first electrode 130a and the second electrode 130b based on the waveform in Channel C. The processing unit 140 may be configured to generate a control signal to cause a corresponding LED 252 to illuminate based on the determined distance. In some embodiments, the medical device 100 may include the LEDs 252, but not the monitor 150, because the LEDs 252 already are capable of informing a user regarding a position of the catheter 132. In other embodiments, the medical device 100 may include both the monitor 150 and the LEDs 252. In further embodiments, the medical device 100 may not include the LEDs 252.

In some embodiments, after the electrode device 200 with the electrodes 250 have been attached to the patient, an imaging (e.g., x-ray imaging) may be performed for the patient to obtain an image. The image may indicate anatomical structure(s) in the patient as well as markers 210 associated with the electrodes 250. In the illustrated example, because the electrodes 250 are aligned next to the markers 210, as shown in FIG. 2C, the positions of the markers 210 may be utilized to indicate, and/or may associate with corresponding electrodes 250. Accordingly, the markers 210 may allow a user to determine an optimal placement position of the catheter 132 to be placed inside the patient. In particular, from the markers 210 captured in the image, the user may then determine which electrode 250 is closest in proximity to a desired target position for the catheter tip (to be inserted and placed inside the patient). The selected one of the electrodes 250 of the electrode device 200 may then function as the first electrode 130a (e.g., TSUI electrode) may then be connected to the processing unit 140 of the device 100 (e.g., via the relay controller 160). After that, a catheter 132 may then be inserted into the patient. The catheter 132 has a catheter electrode 130b (which may be placed at the tip of the catheter), which is also coupled to an input of the device 100. The device 100 with the first electrode 130a and second electrode 130b (e.g., catheter electrode) may then be used to determine positions and placement of the catheter 132.

In some embodiments, the selection of which of the electrodes 250 on the electrode device 200 to function as the first electrode 130a may be achieved via a user interface at the device 100 that operates the relay controller 160. For example, if the electrode device 200 has six electrodes 250, and electrode 250 number 4 is selected as the one to use as the first electrode 130a (e.g., based on a user viewing an imaging showing the positions of the electrodes 250 with respect to a patient's anatomy), the user may operate the user interface to cause the relay controller 160 to communicatively connect electrode number 4 with the processing unit 140 of the medical device 100. The user interface may be a keyboard, a touch screen, one or more buttons, one or more switches, etc. The user interface may be located at, or may be communicatively coupled to, the electrode device 200, to the relay 160, to the processing unit 140, or any combination of the foregoing.

FIG. 2D shows an example of a radiograph illustrating the electrode device 200 coupled to the patient. The radio-opaque markers 210 and the electrodes 250 are visible in the image. The image also shows the third electrode 130c coupled to the patient, and the catheter 132 inserted inside the patient.

In the above embodiments, the electrode device 200 includes both the markers 210 and the electrodes 250. In other embodiments, the electrodes 250 themselves may be radiopaque (in which case, the electrode device 200 may not include the markers 210). In one implementation, parts of the electrodes 250 may be made from an radio-opaque material. In another implementation, a radio-opaque marker may be attached to each of the electrodes 250.

In further embodiments, a marker device (without any electrode) may be utilized to determine a desired position for placement of the first electrode 130a. For example, the marker device may include a patch for securement against a patient, and a plurality of markers. The markers may be radio-opaque markers. During use, the markers of the marker device are imaged to determine which of the markers is closest in position with respect to the target position for the catheter tip. After a marker has been selected, the first electrode 130a may then be placed on the patient in association with the selected marker. For example, the first electrode 130a may be placed next to the selected marker, or at the same location as the selected marker. In such embodiments, the device 100 does not include the relay controller 160, and the first electrode 130a may be implemented without using the electrode device 200 having multiple electrodes 250. For example, in such cases, the first electrode 130a may be a single electrode implemented on a patch for detachably coupling to a patient.

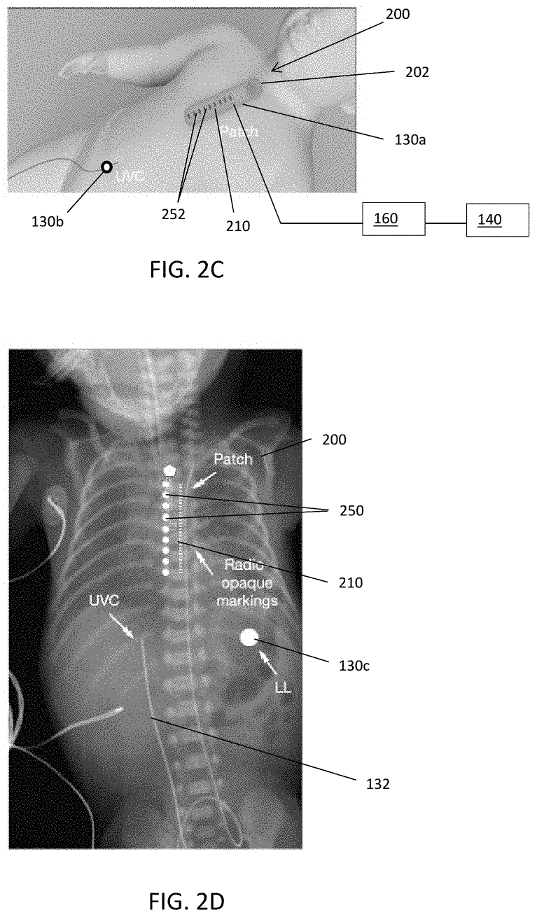

FIG. 3A illustrates placement of electrodes in standard ECG setup. As shown in the figure, in a standard ECG setup, a first electrode 300a is placed at a right arm, a second electrode 300b is placed at the left arm, and a third electrode 300c is placed at the lower chest or leg.

FIG. 3B illustrates placement of the electrodes 130a-130c of the device 100 in accordance with some embodiments. As shown in the figure, the first electrode 130a (the exterior surface electrode) is placed at a chest of a patient, the second electrode 130b is at a catheter, and the third electrode 130c is placed at the lower chest or leg.

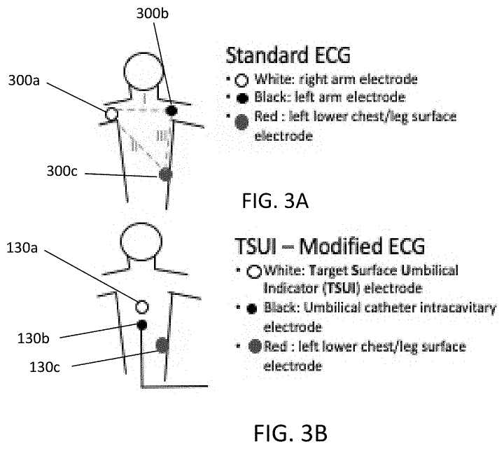

FIGS. 4A-4D below illustrates surface mapping in a patient and operation of the medical device 100. The recorded signals are interpreted by the processing unit 140, which generates graphic visualizing the interpreted signals. The graphic is displayed by the monitor 150. In the illustrated example, the graphic includes three waveforms corresponding to the three respective channels defined among the electrodes 130a-130c. The first electrode 130a is placed at the midline 1 cm below the intermammary line (nipple line). In some embodiments, the first electrode 130a may be a surface electrode implemented by modifying a lead of a cardiac monitoring device. The second electrode 130b is coupled to the catheter 132. In some embodiments, the second electrode 130b may be manufactured with the catheter 132. In other embodiments, the second electrode 130b may be attached to the catheter 132 after the catheter 132 is manufactured. Also, in some embodiments, the second electrode 130b may be connected to the catheter line (e.g., UVC line) via an adapter. For example, a lead functioning as the second electrode 130b may be connected to the UVC line via a Johans adapter or similar adapter. The third electrode 130c is placed on the left leg or left mid axillary line below the heart. Channel A displays Lead II where the waveform represents the difference between the first electrode 130a and the third electrode 130c. Channel B displays Lead III where the waveform represents the difference between the second electrode 130b (e.g., intracavitary electrode) and the third electrode 130c. Channel C displays Lead I where the waveform represents the biopotential difference between the second electrode 130b (e.g., intracavitary electrode) and the first electrode 130a. In the illustrated embodiments, a Channel C “flipping” method is used to identify the proximity and location of the catheter tip relative to a specific target surface electrode.

FIGS. 4A-4D illustrates the catheter 132 of the device 100, and a process for positioning the tip of the catheter 132. In the illustrated example, the catheter 132 is inserted from an umbilical insertion site, and is advanced to reach an IVC within the thoracic region. In other embodiments, the catheter 132 may be other types of catheter, and may be inserted from other sites and/or may be advanced to reach different regions in the patient.

As shown in FIG. 4A, while the catheter 132 with the catheter electrode 130b is at the lower abdominal inside the patient, the ECG trace in Channel C will have a relatively high amplitude due to the long distance with respect to the first electrode 130a (external or surface electrode). Also, the ECG trace in Channel B will have a relatively low amplitude (e.g., zero amplitude) due to the relatively close distance with respect to the third electrode 130c placed at the lower abdomen or leg.

As the catheter 132 is being moved up inside the patient, the ECG traces in Channel B and Channel C change. As shown in FIG. 4B, the ECG trace in Channel C reduces in amplitude due to the second electrode 130b (the catheter electrode) being moved closer towards the first electrode 130a. At the same time, the ECG trace in Channel B increases in amplitude due to the second electrode 130b (the catheter electrode) being moved further from the third electrode 130c at the lower abdomen or leg.

As shown in FIG. 4C, when the catheter 132 is placed directly beneath the first electrode 130a (i.e., at the target position), the ECG trace in Channel C has the lowest amplitude (e.g., zero amplitude), and the ECG trace in Channel B increases even further (compared to that in FIG. 4B).

As shown in FIG. 4D, if the catheter 132 is accidentally moved further up so that it is away from the target position, the ECG trace in Channel C will have an increase in amplitude, and the ECG trace in Channel B will also have an increase in amplitude (compared to those in FIG. 4C). The ECG trace in Channel C has a sign that is “flipped” compared to that in FIGS. 4A-4B. This is because the second electrode 130b has now moved from one side of the first electrode 130a to the opposite side of the first electrode 130a. Accordingly, the ECG trace(s) from the device 100 may be used to assist positioning of the catheter 132 inside the patient.

As illustrated in the above embodiments, cardiac electrophysiology may be utilized to identify the location of electrodes in reference to each other using the heart as a natural “signal generator”.

In some embodiments, a user of the device 100 may decide that the catheter has been desirably placed by viewing the ECG trace in Channel C. In other embodiments, the device 100 may automatically detect that the catheter has been desirably placed based on the minimal amplitude of the ECG trace in Channel C. In such cases, the device 100 may provide a signal to inform the user. For example, the device 100 may provide an audio signal via a speaker, and/or a visual indicator (e.g., a LED indicator, a displayed graphic, etc.), to inform the user that the catheter has been placed at a desired position. In some embodiments, the processing unit 140 may be configured to determine that the catheter has been desirably placed by monitoring the amplitude of the waveform for Channel C (defined between the first and second electrodes 130a, 130b). If the waveform reaches a minimum amplitude, then the processing unit 140 may generate a signal to inform a user that the catheter has been desirably placed inside the patient.

After the initial positioning is completed, a radiograph may be acquired to visualize catheter placement in regard to internal anatomical landmarks to confirm that the catheter 132 has been desirably placed at the target position. In some cases, by observing radio-opaque markings (e.g., markers attached to the patient, such as those described with reference to FIGS. 2C-2D), the caregiver can perform any finer position adjustments if needed.

After the catheter 132 has been desirably positioned in the patient, and its position has been confirmed, the catheter 132 may then be fixed in position with respect to the patient. In some embodiments, the device 100 of FIG. 2A may also be used to monitor the placed catheter 132, so that the device 100 can alarm a user if the catheter 132 is accidentally displaced. For example, if the ECG trace in Channel C has an increase in amplitude, then it indicates that the catheter 132 has been displaced. In such cases, the device 100 of FIG. 2 may provide a signal to inform the user. For example, the device 100 may provide an audio signal via a speaker, and/or a visual indicator (e.g., a LED indicator, a displayed graphic, etc.).

In some embodiments, the processing unit 140 of the device 100 is configured to analyze Channel C to automatically and continuously determine a position of the second electrode 130b (e.g., catheter electrode) with respect to the first electrode 130a (e.g., surface electrode), during placement of the catheter 132 and/or after placement of the catheter 132 in the patient.

In some embodiments, the processing unit 140 may include an analysis unit configured to receive signals from the ports 110. The analysis unit is configured to identify the level of the patch at which ECIF occurs and provide notifications and alerts regarding the location of the tip and in the event of tip migration. In some embodiments, the analysis unit is configured to determine a distance between the first and second electrodes 130a, 130b, and generate a signal to indicate such distance. The analysis unit is configured to repeatedly determine the distance between the first and second electrodes 130a, 130b, and to repeatedly generate the signal to indicate the distance, thereby providing real-time information to a user of the device 100 regarding a placement of the catheter 132 with respect to a target location inside the patient.

In some embodiments, a part of the medical device 100 described herein may be implemented using a ECG monitor. For example, the ECG monitor may display QRS complexes with P-waves. A small QRS indicates that the catheter is below the diaphragm and possibly in the liver or spleen. An inversion of the QRS axis is presumed to indicate that the tip has passed the beyond midline into the spleen. The appearance of a tall positive P-wave will indicate that the catheter tip is at the right atrium level. In the event of a tall positive P-wave, the UVC may then be withdrawn until the P-wave returned to normal size.

The medical devices and methods described herein provide continuous and reliable UVC placement assistance and/or UVC migration monitoring. The devices and methods can not only provide real-time guidance for optimal placement, but also can provide a 24/7 non-invasive monitoring of the position of catheters, such as UVC, umbilical artery catheter (UAC), etc.

As shown in the above embodiments, the channel C (defined between electrodes 130a, 130b) provides distinct signal changes to indicate whether the catheter 132 carrying the second electrode 130b is below (positive deflection), above (flip to negative deflection), and at the same level of the first electrode 130a (zero deflection), such as those shown in FIGS. 4A-4D. These features allow for simple, reliable, and real-time location detection as well as the ability to monitor placement and position of catheter 132 using an alarm. The use of the Channel C “flipping” method to identify the proximity and location of the catheter tip relative to a specific target surface is believed to be novel and inventive.

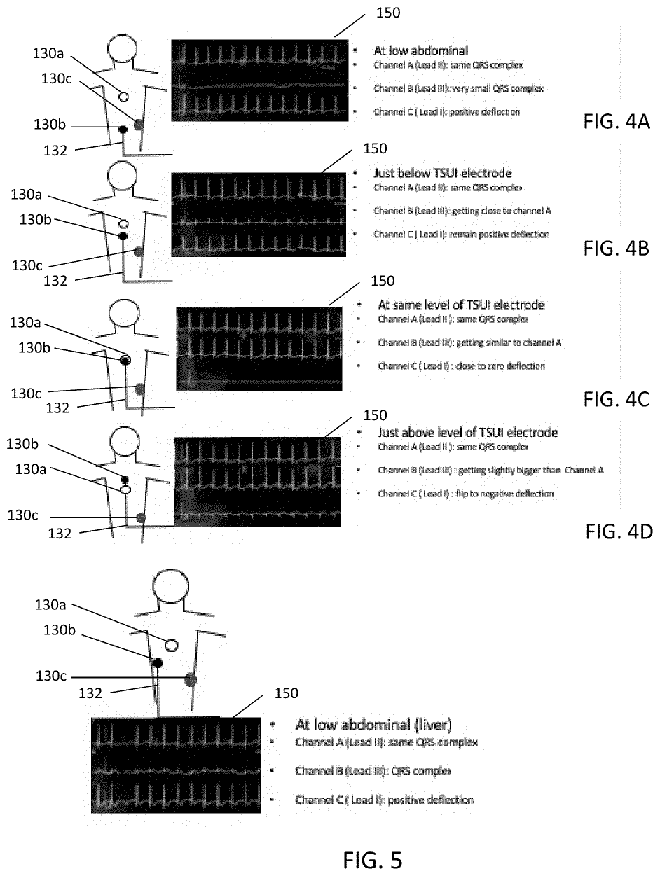

It should be noted that the device 100 and technique described herein are not limited to determining positions of internal electrode along the path shown in FIGS. 4A-4D. In other embodiments, the device 100 and technique described herein may be applied to determine and/or monitor position of internal electrode at other locations in a human body. FIG. 5 illustrates the catheter 132 with the second electrode 130b placed at lower abdomen (e.g., at or near a liver) inside a patient. The signals between the various channels are displayed by the monitor 150 for visualization by a user.

The devices and methods described herein are advantageous because they allow catheter placement to become a straightforward and accurate process that requires only a single radiograph. The ECG tracings will also enable a user to monitor UVC catheter placement more closely, thereby potentially reducing risks associated with the procedure.

The devices and methods described herein are not limited to monitoring UVC placement and positions, and may be applied for other types of catheters (e.g., any catheter type: brand, number of lumens, or diameter), or other objects for placement inside anywhere in a patient's body.

In other embodiments, instead of umbilical venous catheters, the device 100 and technique described herein may be utilized to determine and/or monitor positions of other types of catheters inside patients. For examples, in other embodiments, the device 100 and technique described herein may be utilized to determine and/or monitor positions of biopsy catheters, diagnostic catheters (e.g., catheters with imaging capability), implant delivery catheters, treatment catheters, drug delivery catheters, etc.

Also, in other embodiments, instead of catheters, the device 100 and technique described herein may be utilized to determine and/or monitor positions of other types of medical devices inside patients. For example, in other embodiments, the device 100 and technique described herein may be utilized to determine and/or monitor positions of implants, surgical tools, diagnostic devices, treatment devices, etc., placed inside patients.

Furthermore, in other embodiments, the medical device 100 is not limited to having all of the components shown in FIG. 2A, and may have only a subset of the components shown in FIG. 2A. For example, in other embodiments, the medical device 100 may include only the first electrode 130a, the lead 120a, and the relay 160. In further embodiments, the medical device 100 may include only the electrode device 200 shown in FIG. 2C. In other embodiments, the medical device 100 may include only the processing unit 140. The processing unit 140 may include a signal interpreter for interpreting signals from the electrodes 130a-130c, an analyzer for analyzing the signals to determine a position of the second electrode 130b with respect to the first electrode 130a. In still further embodiments, the medical device 100 may include the first electrode 130a (which may be implemented using the electrode device 200), the second electrode 130b, the third electrode 130c, the relay 160, the processing unit 140, or any combination of the foregoing.

In addition, in some embodiments, at least a part of the medical device 100 may be implemented using a NICU monitor.

Also, in further embodiments, the device 100 may not include the third electrode 103c and the third lead 120c. In such cases, the device 100 may not include the channel A and the channel B.

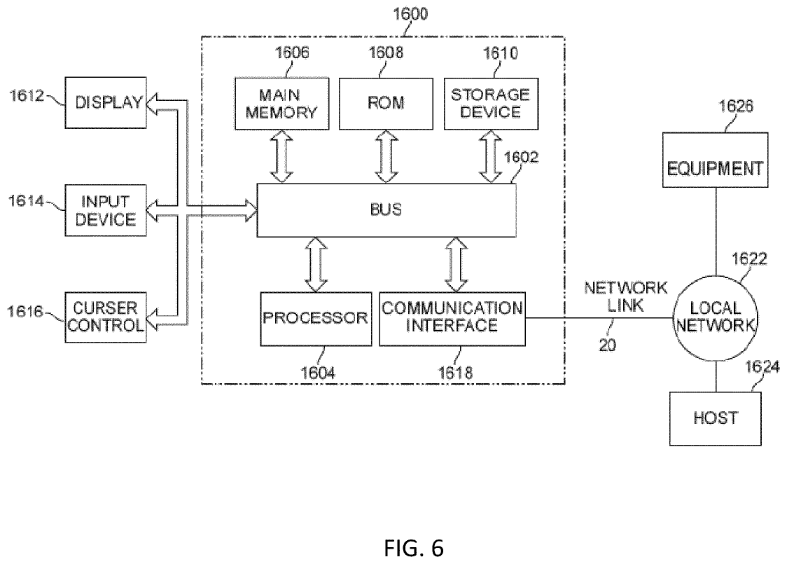

FIG. 6 is a block diagram illustrating an embodiment of a specialized processing system 1600 that can be used to implement various embodiments described herein. For example, the processing system 1600 may be configured to implement the device 100, or one or more components of the device 100, in accordance with some embodiments. For example, in some embodiments, the processing system 1600 may be used to implement the processing unit 140. The processing system 1600 may also be an example of any processor described herein.

Processing system 1600 includes a bus 1602 or other communication mechanism for communicating information, and a processor 1604 coupled with the bus 1602 for processing information. The processor system 1600 also includes a main memory 1606, such as a random access memory (RAM) or other dynamic storage device, coupled to the bus 1602 for storing information and instructions to be executed by the processor 1604. The main memory 1606 also may be used for storing temporary variables or other intermediate information during execution of instructions to be executed by the processor 1604. The processor system 1600 further includes a read only memory (ROM) 1608 or other static storage device coupled to the bus 1602 for storing static information and instructions for the processor 1604. A data storage device 1610, such as a magnetic disk or optical disk, is provided and coupled to the bus 1602 for storing information and instructions.

The processor system 1600 may be coupled via the bus 1602 to a display 167, such as a flat panel, for displaying information to a user. An input device 1614, including alphanumeric and other keys, is coupled to the bus 1602 for communicating information and command selections to processor 1604. Another type of user input device is cursor control 1616, such as a mouse, a trackball, or cursor direction keys for communicating direction information and command selections to processor 1604 and for controlling cursor movement on display 167. This input device typically has two degrees of freedom in two axes, a first axis (e.g., x) and a second axis (e.g., y), that allows the device to specify positions in a plane.

In some embodiments, the processor system 1600 can be used to perform various functions described herein. According to some embodiments, such use is provided by processor system 1600 in response to processor 1604 executing one or more sequences of one or more instructions contained in the main memory 1606. Those skilled in the art will know how to prepare such instructions based on the functions and methods described herein. Such instructions may be read into the main memory 1606 from another processor-readable medium, such as storage device 1610. Execution of the sequences of instructions contained in the main memory 1606 causes the processor 1604 to perform the process steps described herein. One or more processors in a multi-processing arrangement may also be employed to execute the sequences of instructions contained in the main memory 1606. In alternative embodiments, hard-wired circuitry may be used in place of or in combination with software instructions to implement the various embodiments described herein. Thus, embodiments are not limited to any specific combination of hardware circuitry and software.

The term “processor-readable medium” as used herein refers to any medium that participates in providing instructions to the processor 1604 for execution. Such a medium may take many forms, including but not limited to, non-volatile media, volatile media, and transmission media. Non-volatile media includes, for example, optical or magnetic disks, such as the storage device 1610. A non-volatile medium may be considered an example of non-transitory medium. Volatile media includes dynamic memory, such as the main memory 1606. A volatile medium may be considered an example of non-transitory medium. Transmission media includes coaxial cables, copper wire and fiber optics, including the wires that comprise the bus 1602. Transmission media can also take the form of acoustic or light waves, such as those generated during radio wave and infrared data communications.

Common forms of processor-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a processor can read.

Various forms of processor-readable media may be involved in carrying one or more sequences of one or more instructions to the processor 1604 for execution. For example, instructions may be stored in a portable storage device, such as a USB or a memory disk. The storage device may be detachably coupled to the processing system 1600 for transferring the instructions to the processing system 1600. As another example, the instructions may be stored in a “cloud”. In such cases, the instructions may be downloaded from a server to the processing system 1600. As another example, the instructions may initially be carried on a magnetic disk of a remote computer. The remote computer can load the instructions into its dynamic memory and send the instructions over a network, such as a wireless network a telephone line, etc. A communication device, such as a network interface or a modem, local to the processing system 1600 can receive the data on the network, and provide the data on the bus 1602. The bus 1602 carries the data to the main memory 1606, from which the processor 1604 retrieves and executes the instructions. The instructions received by the main memory 1606 may optionally be stored on the storage device 1610 either before or after execution by the processor 1604.

The processing system 1600 also includes a communication interface 1618 coupled to the bus 1602. The communication interface 1618 provides a two-way data communication coupling to a network link 1620 that is connected to a local network 1622. For example, the communication interface 1618 may be an integrated services digital network (ISDN) card or a modem to provide a data communication connection to a corresponding type of telephone line. As another example, the communication interface 1618 may be a local area network (LAN) card to provide a data communication connection to a compatible LAN. Wireless links may also be implemented. In any such implementation, the communication interface 1618 sends and receives electrical, electromagnetic or optical signals that carry data streams representing various types of information.

The network link 1620 typically provides data communication through one or more networks to other devices. For example, the network link 1620 may provide a connection through local network 1622 to a host computer 1624 or to equipment 1626. The data streams transported over the network link 1620 can comprise electrical, electromagnetic or optical signals. The signals through the various networks and the signals on the network link 1620 and through the communication interface 1618, which carry data to and from the processing system 1600, are exemplary forms of carrier waves transporting the information. The processing system 1600 can send messages and receive data, including program code, through the network(s), the network link 1620, and the communication interface 1618.

Although particular features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications and equivalents.

What is claimed is:

1. A medical method performed by a medical device, comprising:
generating a ECG tracing using a first electrode placed on a surface of a patient and a second electrode at a catheter inside the patient; and
outputting the ECG tracing for presentation to a user;
wherein the first electrode is fixed in position with respect to the patient; and
wherein an amplitude of the ECG tracing corresponds with a relative distance between the first electrode and the second electrode.

2. The method of claim 1, wherein the catheter comprises an umbilical venous catheter.

3. The method of claim 1, wherein the patient comprises a pediatric patient.

4. The method of claim 1, wherein the amplitude of the ECG tracing is equal to zero or has a minimum value when the second electrode is directly beneath the first electrode.

5. The method of claim 1, wherein the ECG tracing is outputted for display on a screen.

6. The method of claim 1, wherein the first electrode is located above a target position for the second electrode.

7. The method of claim 1, further comprising generating an audio signal when the second electrode is underneath the first electrode.

8. The method of claim 1, further comprising repeating the act of generating and the act of outputting while the catheter is being positioned inside the patient.

9. The method of claim 8, wherein the repeating is performed to provide a real-time indication of a position of the catheter with respect to the first electrode.

10. The method of claim 1, further comprising repeating the act of generating and the act of outputting after the catheter has been placed at a target position inside the patient.

11. The method of claim 10, wherein the repeating is performed to allow monitoring of the placed catheter at the target position.

12. The method of claim 10, further comprising generating an audio signal when the catheter is displaced from the target position.

13. A medical device comprising:
a first electrode configured for placement on a surface of a patient;
a second electrode configured for coupling with a catheter; and
a processing unit configured to generate a ECG tracing using the first electrode on the surface of the patient and the second electrode inside the patient.

14. The medical device of claim 13, further comprising a screen for displaying the ECG tracing.

15. The medical device of claim 13, wherein the processing unit is a part of a ECG device.

16. The medical device of claim 13, wherein the catheter comprises an umbilical venous catheter, and the second electrode is configured for coupling with the umbilical venous catheter.

17. The medical device of claim 13, wherein the processing unit is configured to generate the ECG tracing with an amplitude that is equal to zero or has a minimum value when the second electrode is directly beneath the first electrode.

18. The medical device of claim 13, further comprising a screen, wherein the processing unit is configured to output the ECG tracing for display on the screen.

19. The medical device of claim 13, wherein the first electrode is configured for placement above a target position for the second electrode.

20. The medical device of claim 13, further comprising a speaker configured to provide an audio signal when the second electrode is underneath the first electrode.

21. The medical device of claim 13, wherein the processing unit is configured to generate additional ECG tracing(s) while the catheter is being positioned inside the patient.

22. The medical device of claim 21, wherein the additional ECG tracing(s) provides a real-time indication of a position of the catheter with respect to the first electrode.

23. The medical device of claim 13, wherein the processing unit is configured to generate additional ECG tracing(s) after the catheter has been placed at a target position inside the patient.

24. The medical device of claim 23, wherein the additional ECG tracing(s) provides monitoring of the placed catheter at the target position.

25. The medical device of claim 23, further comprising a speaker configured to provide an audio signal when the catheter is displaced from the target position.

26. The medical device of claim 23, wherein the first electrode is on a pad with an adhesive surface and a plurality of markers.

27. The medical device of claim 26, wherein first electrode is located on the pad in association with one of the markers.

28. The medical device of claim 26, wherein the markers are in a single row.

29. The medical device of claim 26, wherein the markers are arranged in rows and columns.

30. A medical device comprising:
a pad having an adhesive surface;
a plurality of markers at the pad; and
an external electrode in positional correspondence with a selected one of the markers, wherein the external electrode is configured to couple with a lead extending from an apparatus, the apparatus having a processing unit.

31. The medical device of claim 30, wherein the markers are in a single row.

32. The medical device of claim 30, wherein the markers are arranged in rows and columns.

33. The medical device of claim 30, further comprising a catheter electrode configured to couple with another lead extending from the apparatus.

34. The medical device of claim 30, further comprising the apparatus.

35. The medical device of claim 34, wherein the apparatus comprises a ECG device.

36. A medical device comprising:
a pad having an adhesive surface;
a plurality of electrodes coupled to the pad; and
a plurality of terminals respectively at, or electrically connected to, the plurality of electrodes;
wherein each of the terminals in the plurality of terminals is capable of being detachably coupled to a lead extending from an apparatus, the apparatus having a processing unit.

37. The medical device of claim 36, wherein the electrodes in the plurality of electrodes are in a single row.

37. The medical device of claim 36, wherein the electrodes in the plurality of electrodes are arranged in rows and columns.

39. The medical device of claim 36, further comprising a catheter electrode configured to couple with another lead extending from the apparatus.

40. The medical device of claim 36, wherein the medical device further includes the apparatus.

41. The medical device of claim 40, wherein the apparatus comprises a ECG device.

42. The medical device of claim 36, wherein the electrodes are radiopaque.

43. The medical device of claim 36, wherein the electrodes are coupled to respective markers.

44. A medical method comprising:
placing a pad on a surface of a patient, the pad having a plurality of markers;
taking an image of the patient, the image indicating an internal anatomical structure of the patient and the plurality of markers;
coupling an electrode with an apparatus via a lead, wherein the electrode is in positional correspondence with one of the plurality of markers that is the closest in position with respect to the internal anatomical structure of the patient as they appear in the image.

45. The method of claim 44, wherein the markers are in a single row.

46. The method of claim 44, wherein the markers are arranged in rows and columns.

47. The method of claim 44, wherein the apparatus comprises a ECG device.

48. The method of claim 44, further comprising using the apparatus to monitor a position of a catheter while the catheter is being positioned within the patient.

49. The method of claim 48, wherein the catheter comprises an umbilical venous catheter.

50. The method of claim 44, wherein the patient comprises a pediatric patient.
